(19) United States
(12) Patent Application Publication
(10) Pub. No.: US 2021/0053748 A1
(43) Pub. Date: Feb. 25, 2021

(54) Package For Delivery of Volatile Substance

(71) Applicant: Car-Freshner Corporation, Watertown, NY (US)

(72) Inventor: Scott Goodrich, Sackets Harbor, NY (US)

(73) Assignee: Car-Freshner Corporation, Watertown, NY (US)

(21) Appl. No.: 16/548,433

(22) Filed: Aug. 22, 2019

(51) Int. Cl. B65D 85/00; B65D 81/24; B65D 81/26

(52) U.S. Cl. CPC B65D 85/70; B65D 81/264; B65D 81/245

(57) ABSTRACT

A package for delivering a fluid volatile substance includes an impermeable shell that defines a reservoir into which a liquid volatile substance, such as oil, may be at least partially filled. A microporous membrane is placed in covering relation to the reservoir to allow emanation through capillarity of the volatile substance. To absorb any liquid that may seep through microporous membrane during use or storage, an oleophilic layer is heat sealed in covering relation to the microporous membrane. Finally, a foil barrier layer is sealed to the shell in covering relation to the microporous membrane. The foil barrier is releasable and once released will activate the volatile substance contained within the reservoir to transition from its liquid phase to a gaseous phase with the vapors carrying the scent of the volatile substance.

### FIELD OF THE INVENTION

The present disclosure is directed generally to a package for delivering a volatile sub stance.

### BACKGROUND

Packages for controlling the emanation of volatile substances, such as oil, are known in the art. For example, packages having a reservoir in which a volatile fluid is contained, a foil barrier to retain the volatile substance within the reservoir until activated, and a membrane cover to absorb and permit emission through evaporation of the volatile substance while preventing the fluid from spilling out of the reservoir before it evaporates. With the prior art packages, however, the membrane often becomes saturated and the fluid drips from the membrane.

Accordingly, there is a need in the art for a volatile emitting package that permits effective evaporation of the volatile substance while preventing the fluid from dripping or otherwise leaking from the package.

### SUMMARY

The present disclosure is directed to a package for delivering a volatile substance.

According to an aspect is a package for delivering a fluid volatile substance, comprising a shell in which the fluid volatile substance is at least partially filled; a microporous membrane positioned in covering relation to the shell; an oleophilic sorbent layer positioned in covering relation to the microporous membrane; and a releasable barrier film positioned in covering relation to the oleophilic sorbent layer.

According to an embodiment, the microporous membrane is composed of TESLIN.

According to an embodiment, the oleophilic sorbent layer is composed of spunbond polypropylene.

According to an embodiment, the barrier film is composed of foil.

According to an embodiment, the oleophilic layer is heat sealed to the microporous membrane.

According to an embodiment, the oleophilic layer is more porous than the microporous membrane.

According to an aspect is a method for manufacturing a package for delivering a fluid volatile substance, comprising the steps of providing a plurality of shells in each of which a volatile liquid substance is at least partially filled; sealing a microporous membrane in covering relation to each shell; sealing an oleophilic sorbent layer in covering relation to the microporous membrane; and sealing a releasable barrier film in covering relation to the oleophilic sorbent layer.

According to an embodiment, the steps of sealing a microporous membrane in covering relation to each shell and sealing an oleophilic sorbent layer in covering relation to the microporous membrane, further comprise the steps of unwinding the microporous membrane in covering relation to each shell, unwinding the oleophilic sorbent layer in covering relation to the microporous membrane, and then applying a heat seal to effect the sealing of the microporous membrane and the oleophilic sorbent layer.

According to an embodiment, following the step of applying a heat seal to effect the sealing of the microporous membrane and the oleophilic sorbent layer, comprising the further steps of: kiss cutting the sorbent and membrane webs outside of the heat sealed area; and rewinding the kiss cut sorbent and membrane webs.

According to an embodiment, the step of sealing a releasable barrier film in covering relation to the oleophilic sorbent layer comprises unwinding the barrier fill in covering relation to the oleophilic sorbent layer and applying a heat seal thereto to create a plurality of sealed shells.

According to an embodiment, the method comprises the further step of die cutting the sealed shells.

These and other aspects of the invention will be apparent from the embodiments described below.

### BRIEF DESCRIPTION OF THE DRAWINGS

The present invention will be more fully understood and appreciated by reading the following Detailed Description in conjunction with the accompanying drawings, in which:

FIG. 1 is a cross-sectional view of a package for delivering a fluid volatile substance, in accordance with an embodiment.

FIG. 2 is a schematic representation of a process for manufacturing a package for delivering a fluid volatile substance, in accordance with an embodiment.

### DETAILED DESCRIPTION OF EMBODIMENTS

The present disclosure describes a package for delivering a fluid volatile substance and a method for manufacturing a package for delivering a fluid volatile substance.

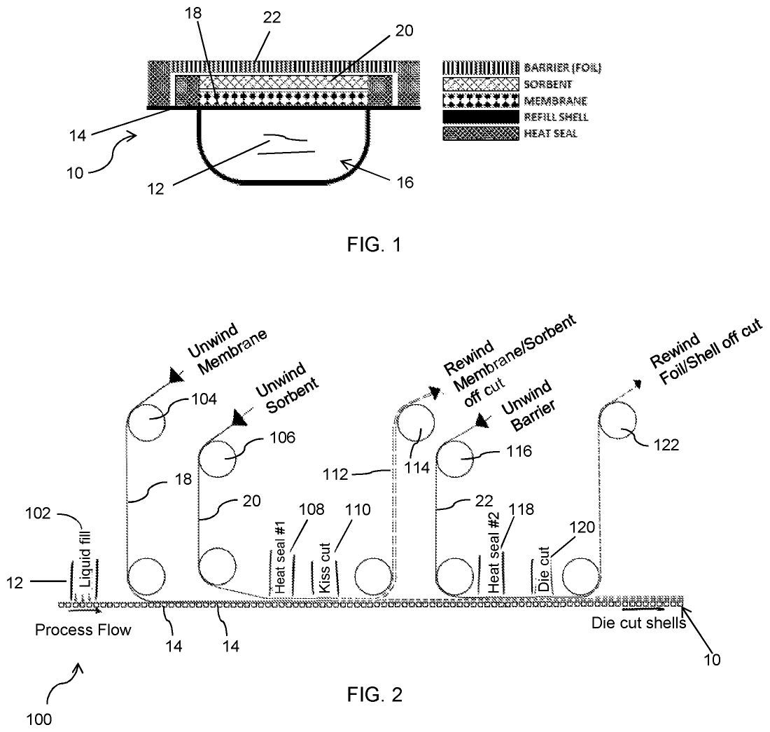

Referring to FIG. 1, in one embodiment, is a package, designated generally by reference numeral 10, for delivering a fluid volatile substance 12. Package 10 comprises an impermeable shell 14 that defines a reservoir 16 in which a liquid volatile substance (e.g., oil) is at least partially filled. A microporous membrane 18, preferably composed of TESLIN® (although other microporous membrane material will also work), is sealed to shell 14 in covering relation to reservoir 16. Microporous membrane 18 permits emanation of the volatile fluid 12 after transitioning into a gaseous phase therethrough, as described in greater detail hereinafter. Covering and sealed to microporous membrane 18 is an oleophilic sorbent layer 20, preferably composed of spunbond polypropylene (although other oleophilic materials will also work), which absorbs any of the liquid volatile substance that may leach through microporous membrane 18 prior to transitioning into a gaseous phase. The final component of package 10 is a barrier layer 22, preferably composed of foil, which is sealed to shell 14 in covering relation to oleophilic sorbent layer 20. Barrier layer 22 seals package 10 and prevents volatile substance 12 from transitioning from liquid phase to gas phase until its release from package 10. Upon release, the volatile substance is free to begin transitioning from its liquid phase to a gaseous phase due to direct exposure to the atmosphere with the vapors carrying the scent from the material composing the volatile substance.

Referring to FIG. 2, a method for manufacturing package 10 is schematically illustrated. A strip or web of shells 14 are fed on an assembly line 100 with a predetermined quantity of fluid volatile substance 12 being injected into reservoirs 16 at a liquid fill station 102. After being filled with fluid volatile substance 12, the process continues with microporous membrane 18 being unwound by an unwinding station 104 and laid on shells 14 in covering relation over reservoirs 16. Next, oleophilic sorbent layer 20 is unwound by an unwinding station 106 and positioned in covering relation to microporous membrane 18. A heat seal device 108 then applies heat and seals the microporous membrane 18 and oleophilic layer 20 together and to the shells 14. After heat sealing, the sorbent and membrane webs are kiss cut outside of the heat sealed area by kiss cutting station 110. The off-cut material of sorbent and membrane 112 are stripped away from the sorbent/membrane/shell web and subsequently rewound at rewind station 114. The barrier 22 is then unwound at an unwinding station 116 and placed in covering relation to the oleophilic layer 20 and a heat seal device 118 then applies heat to seal the barrier 22 to shell 14. The sealed shells 14 are die cut into predetermined quantities/patterns of shells by a die cutting station 120 and complete the package 10. A rewind station 122 rewinds the off-cut foil/shell, while the completed packages 10 are assembled.

In operation, as described above, upon release of the barrier layer 22 from shell 14, the volatile substance 12 begins transitioning from liquid phase to gaseous phase with the vapors carrying the scent of the volatile substance. Due to the permeability of membrane 18 that permits emanation of the volatile substance some quantity of volatile substance 12 in its liquid state may seep through. Oleophilic layer 20 is built into package to absorb any such leaking liquid and prevent it from dripping or otherwise spilling from package 10 upon release of barrier 22. Moreover, the liquid that is absorbed into oleophilic layer 20 will emanate therefrom in vapor form when exposed to air. Furthermore, the oleophilic layer 20 is, as described above, heat sealed to the microporous membrane 18, rather than being heat sealed to the barrier layer 22. This aspect keeps the accumulated volatile substance with the reservoir/membrane sub-assembly, as compared to the oleophilic layer 20 being sealed to the barrier 22. One further aspect is, notably, that the porosity of the oleophilic layer 20 is greater than that of the microporous membrane 18, thereby allowing for greater emanative flux of the volatile substance 12 to avoid any decrease in membrane emanation performance after initially saturated volatile substance has evaporated.

While various embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, embodiments may be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

What is claimed is:

1. A package for delivering a fluid volatile substance, comprising:
a. a shell in which the fluid volatile substance is at least partially filled;
b. a microporous membrane positioned in covering relation to the shell;
c. an oleophilic sorbent layer positioned in covering relation to the microporous membrane; and
d. a releasable barrier film positioned in covering relation to the oleophilic sorbent layer.

2. The package according to claim 1, wherein the microporous membrane is composed of TESLIN.

3. The package according to claim 1, wherein the oleophilic sorbent layer is composed of spunbond polypropylene.

4. The package according to claim 1, wherein the barrier film is composed of foil.

5. The package according to claim 1, wherein the oleophilic layer is heat sealed to the microporous membrane.

6. The package according to claim 1, wherein the oleophilic layer is more porous than the microporous membrane.

7. A method for manufacturing a package for delivering a fluid volatile substance, comprising the steps of:
a. providing a plurality of shells in each of which a volatile liquid substance is at least partially filled;
b. sealing a microporous membrane in covering relation to each shell;
c. sealing an oleophilic sorbent layer in covering relation to the microporous membrane; and
d. sealing a releasable barrier film in covering relation to the oleophilic sorbent layer.

8. The method according to claim 7, wherein the steps of sealing a microporous membrane in covering relation to each shell and sealing an oleophilic sorbent layer in covering relation to the microporous membrane, further comprise the steps of unwinding the microporous membrane in covering relation to each shell, unwinding the oleophilic sorbent layer in covering relation to the microporous membrane creating a sorbent and membrane web, and then applying a heat seal to effect the sealing of the microporous membrane and the oleophilic sorbent layer.

9. The method according to claim 8, following the step of applying a heat seal to effect the sealing of the microporous membrane and the oleophilic sorbent layer, comprising the further steps of:
a. kiss cutting the sorbent and membrane webs outside of the heat sealed area; and
b. rewinding the kiss cut sorbent and membrane webs.

10. The method according to claim 7, wherein the step of sealing a releasable barrier film in covering relation to the oleophilic sorbent layer comprises unwinding the barrier fill in covering relation to the oleophilic sorbent layer and applying a heat seal thereto to create a plurality of sealed shells.

11. The method according to claim 10, comprising the further step of die cutting the sealed shells.
